(19) United States
(12) Patent Application Publication
(10) Pub. No.: US 2004/0116807 A1
(43) Pub. Date: Jun. 17, 2004

(54) Blood vessels wall imaging catheter

(76) Inventors: Roni Amrami, Yokneam (IL); Yoav Kimchy, Haifa (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/686,536

(22) Filed: Oct. 16, 2003

Related U.S. Application Data
(60) Provisional application No. 60/418,712, filed on Oct. 17, 2002.
(60) Provisional application No. 60/424,677, filed on Nov. 8, 2002.

(51) Int. Cl. A61B 6/00

(52) U.S. Cl. 600/436; 600/476

(57) ABSTRACT

A system and method are provided for intravascular nuclear imaging, which is furthermore designed to locate the plaque along the artery and to estimate its thickness. Radiolabeled antibodies directed specific antigens in the atherosclerotic plaque or radiolabeled immunoscintigraphy pharmaceutical, which binds apoptotic cells, have been proposed for detection of vulnerable plaque. Other Radiopharmaceuticals such as Gallium Citrate, radio labeled white blood cells etc. call also be used to illuminate inflammatory processes.

This application derives priority from U.S. Provisional Application No. 60/418,712, filed on Oct. 17, 2002, and XXXXX, filed on Nov. 8, 2002.

### FIELD AND BACKGROUND OF THE INVENTION

The present invention relates to the diagnosis and treatment of cardiac arteries disease, and more particularly, to a system and method for imaging vulnerable plaque in coronary arteries.

Heart attacks seem to occur suddenly and often without warning, yet, the process underlying the event has been going on for many years.

In principle, nuclear imaging is an ideal method to investigate most function processes, mainly perfusion, metabolism and receptor-ligand processes, as it has an exquisite sensitivity being able to identify molecules down to the picomole range. This is unmatched by any other imaging modality. However, extracorporeal nuclear imaging has not been adapted broadly for study of atherosclerosis due to the radiation and safety problems, poor resolution and lack of specificity, and ordinary gamma probes may not be inserted into the arteries.

Today's regular procedure is: If patient medical tests reveal a moderate to severe limitation in blood flow at peak stress versus rest, then the usual procedure is to open the blocked artery. This procedure, called angioplasty with stent placement has evolved to become an extremely advanced procedure in recent years. First, a thin flexible plastic catheter is advanced through an artery at the top of the leg under x-ray until it gets to the heart. Dye is then injected into each of the coronary arteries and a movie is taken in order to determine exactly where and how severe the blockage or blockages are. If there are one or two tight discrete blockages, then a wire with a tightly wrapped tiny balloon can be advanced across the narrowed segment; the balloon is inflated under very high pressures, thereby crushing the cholesterol plaque up against the wall. The balloon is removed, and a tiny metal scaffold wrapped on a balloon, is then advanced into the area and blown up against the wall under high pressure. The balloon is removed, leaving the stent permanently embedded in the wall. The stent helps keep the artery open significantly better than balloon angioplasty alone.

### SUMMARY OF THE INVENTION

The present invention provides a system and method for intravascular nuclear imaging, which is furthermore designed to locate the plaque along the artery and to estimate its thickness. Radiolabeled antibodies directed specific antigens in the atherosclerotic plaque or radiolabeled immunoscintigraphy pharmaceutical, which binds apoptotic cells, have been proposed for detection of vulnerable plaque. Other Radiopharmaceuticals such as Gallium Citrate, radio labeled white blood cells etc. call also be used to illuminate inflammatory processes.

Implementation of the methods and systems of the present invention involves performing or completing selected tasks or steps manually, automatically, or a combination thereof. Moreover, according to actual instrumentation and equipment of preferred embodiments of the methods and systems of the present invention, several selected steps could be implemented by hardware or by software on any operating system of any firmware or a combination thereof. For example, as hardware, selected steps of the invention could be implemented as a chip a circuit. As software, selected steps of the invention could be implemented as a plurality of software instructions being executed by a computer using any suitable algorithms. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates electrons energy wpectra measurements inside the blood vessel, in accordance with the present invention;

FIG. 2 schematically illustrates a cross sectional view of an imaging apparatus inside a blood vessel wall, in accordance with the present invention;

FIG. 3 schematically illustrates a general view of the detector inside the artery cross section and a timing coded detector block diagram, in accordance with the present invention;

FIG. 4 schematically illustrates a light detector signal pair from the two fiber's ends, in accordance with the present invention;

FIG. 5 schematically illustrates a radiation interaction point at the fiber's side length surface verses the measured light arrival time difference from the two fiber's ends, in accordance with the present invention;

FIG. 6 schematically illustrates a fiber timing coded reflected detector's (cross-section) flow diagram, in accordance with the present invention;

FIG. 7 schematically illustrates a general view of the detector inside the artery cross section and the coded detector inside the artery cross section, in accordance with the present invention;

FIG. 8 schematically illustrates a signal processing flow block diagram, in accordance with the present invention;

FIG. 9 schematically illustrates a multi fiber detector assembly cross section threaded on a guide wire, in accordance with the present invention;

FIG. 10 is a wavelength spectra, in accordance with the present invention; and

FIG. 11 is a position vs. intensity ID graph, indicating possible positions of venerable plaque along the path of the color coded optic fiber, in accordance with the present invention.

### DESCRIPTION OF THE PREFERRED EMBODIMENTS

The present invention is of a system and method for intravascular nuclear imaging, which is furthermore designed to locate the plaque along the artery and to estimate its thickness. Radiolabeled antibodies directed specific antigens in the atherosclerotic plaque or radiolabeled immunoscintigraphy pharmaceutical, which binds apoptotic cells, have been proposed for detection of vulnerable plaque. Other Radiopharmaceuticals such as Gallium Citrate, radio labeled white blood cells etc. call also be used to illuminate inflammatory processes.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

### First Embodiment

The present invention, of intravascular nuclear imaging, enables one to identify the previously undetectable areas in coronary arteries most likely to cause high risk situations (Venerable Plaque). The system maps the arterial walls of the coronary arteries on the basis of radiolabeled Immunoscintigraphy pharmaceutical activity variations based on the known fact that some radiopharmaceuticals show increased concentration in areas of inflammatory processes, which are thought to correlate with the vulnerability of the coronary artery to eruption. It is speculated that these plaques are the cause of 60-80% of Acute Cardiac Syndrome (ACS).

Thus, one might image radiolabeled antibodies directed specific antigens in the atherosclerotic plaque or radiolabeled Immunoscintigraphy pharmaceutical, which binds apoptotic cells and has been proposed for detection of vulnerable plaque. Other Radiopharmaceuticals such as Gallium Citrate, radio labeled white blood cells etc. call also be used to illuminate inflammatory processes.

The disclosed invention will enable the physician to have a good assessment of wall thikness and more importantly of potential wall thinning, which may relate to vulnerable plaques. For this, before the above procedure the patient will be injected with a radioisotope, which may or may not be bonded to a pharmaceutical that targets inflammatory processes (Gallium Citrate, Radio labeled white blood cells etc), which have been proposed for detection of vulnerable plaque. Examples of such radioisotopes include Pt, Ti, Tc, Ga, In, I etc.

After the injection, the blood vessel surrounding irradiates radioactive particles (such as beta, electrons or positrons). As those particles pass through the blood vessel wall they arc absorbed or attenuated (weakened) at differing levels creating a matrix or profile of particles of different energies.

The equation that is used to calculate the energy loss of a beta particle is:

$$\frac{ⅆ E}{ⅆ x} = \frac{2 {Π}_{q}^{4} NZ \times {( 3 \times {10}^{9} )}^{4}}{{E}_{m} {ρ}^{2} \times {( 1.6 \times {10}^{- 6} )}^{2}} [ ln ( \frac{{E}_{m} {E}_{k} {ρ}^{2}}{{I}^{2} ( 1 - {ρ}^{2} )} ) - {ρ}^{2} ] \frac{Me V}{c m}$$

Where

q=charge on the electron, 1.6×10⁻C,

M=rest mass of the ionizing particle, grams,

N=number of absorber atoms per cm³,

Z=atomic number of the absorber,

NZ=number of absorber electrons per cm3=3.88×1020 for air at 0° and 76 cmHg

β=v/c

Eₘ=energy equivalent of electron mass, 0.51 MeV

Eₖ=Kinetic energy of the beta particle, MeV

I=mean excitation and ionization potential of absorbing atoms, MeV

I=8.6×10⁻⁵ erg for air, for other substances, I=1.35×10⁻⁵ Z.

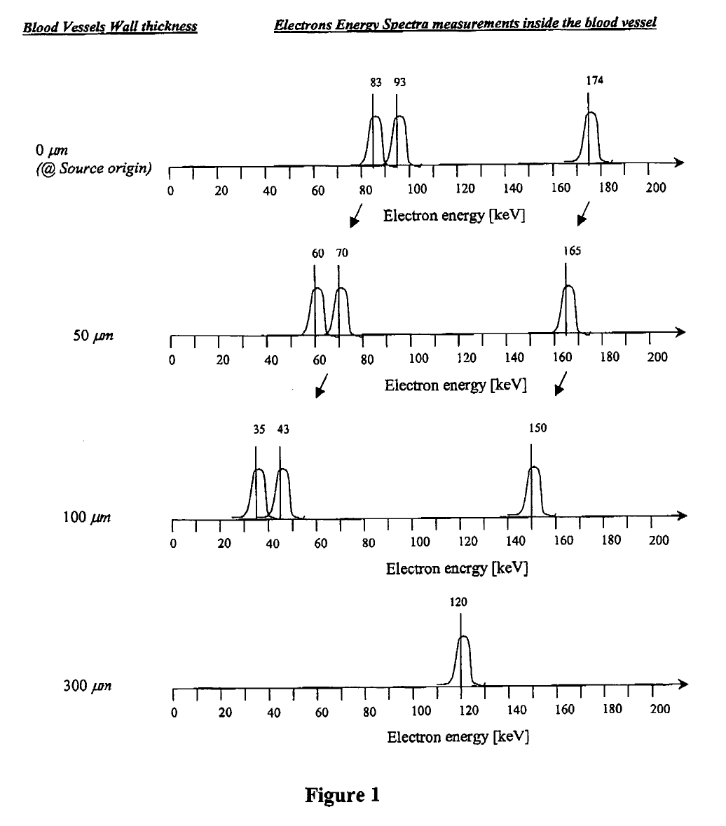

Referring now to the drawings, FIG. 1 illustrates Blood Vessels Wall thickness and Electrons Energy Spectra measurements inside the blood vessel, measured in accordance with the present invention.

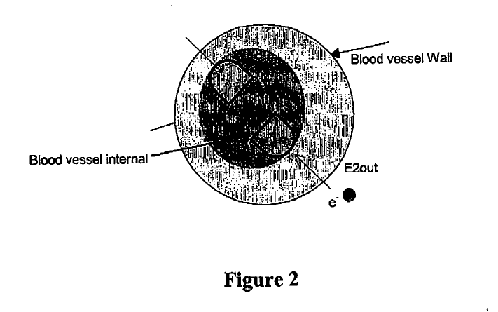

Referring further to the drawings, FIG. 2 schematically illustrates schematically illustrates a cross sectional view of an imaging apparatus inside a blood vessel wall, in accordance with the present invention.

A radiation detector detects this particle profile. By moving one or few detectors and/or using detector array an anatomical and/or physiological image can be created.

Each profile is subdivided spatially (divided into partitions) by the detectors and fed into individual channels. Each profile is then backwards reconstructed (or “back projected”) by a dedicated computer into a two-dimensional image of the “slice” that was scanned.

The blood vessel wall thickness measurement resolution is determined by electrons kinetic energy, as shown in Table 1, below, and detector energy resolution.

TABLE 1

| | Blood Vessels Wall Thickness |
|---|---|
| Electron Kinetic Energy | Measurement Resolution |
| [keV] | [□m] |
| tens | tens |
| hundreds | hundreds |
| thousands | thousands |

A high wall thickness resolution can be achieved by using positrons spectra (for example from isotope ¹⁸F).

This invention measures the blood vessel wall thickness at a high level of accuracy using advanced computer software.

The system presents the energetic active part after attenuated by the blood vessel wall and can be use with and/or on top of the imaging modality information (CT MRI Fluoro etc.). The physician uses this information for therapeutic and/or procedure planning as well as to validate the placement of a device to be within the active areas.

For the procedure, the patient in the CAT lab is first injected with the suitable radioisotope. Then, during catheterization, a catheter is passed along the suspected coronary artery to the distal part, then the balloon caring the radiation detectors is slightly inflated so that it touches the arterial walls. Then, the catheter is pulled.

Radiation counts together with their energy spectrum, and the position of the catheter are processed to form a 3D reconstructed image of the wall thickness as the catheter is moved along the coronary artery.

This is achieved since the energy spectrum of the particles is relative to the amount of attenuation that the particles experienced and this in turn relates to the vessel wall anatomy. Having a multitude of detectors around the circumference of the catheter enables imaging the whole circumference of the blood vessel wall thickness as the catheter is pulled by registering the electron spectral energy for every detector on the circumference in relation to the ID position along the vessel path.

### EXAMPLES

Reference is now made to the following examples, which together with the above description illustrate the invention in a non-limiting fashion. The following are examples of Example for a radiation detector that can be used.

### Example 1

Gas Filled Detectors with Gas such as CO₂ CH₄

Ionization chamber

Proportional chamber

Geiger chamber

### Example 2

Scintillation Detectors

Organic scintillators crystals and liquids

C₁₄H₁₀, C₁₄H₁₂, C₁₀H₈ etc.

Plastics

NE102A, NE104, NE110

Pilot U

Inorganic scintillators

NaI

CsI

BGO

LSO

YSO

BaF

ZnS

ZnO

CaWO₄

CdWO₄

### Example 3

Scintillator coupling:

Photomultiplier Tube (PMT)

Side-on type

Head-on type

Hemispherical type

Position sensitive

Microchannel Plate-Photomultiplier (MCP-PMTs)

Electron multipliers

Photodiodes (& Photodiodes Arrays)

Si photodiodes

Si PIN photodiodes

Si APD

GaAs(P) photodiodes

GaP

CCD

### Example 4

Solid-State Detectors: N-type, P-type, PIN-type Pixellated or Unpixellated

a-Ge, Ge, Ge(Li)

a-Si, Si, Si(Li)

CdTe

CdZnTe

CdSe

CdZnSe

HgI₂

TlBrI

GaAs

InI

GaSe

Diamond

TlBr

PbI₂

InP

ZnTe

HgBrI

a-Se

BP

GaP

CdS

SiC

AlSb

PbO

BiI₃

ZnSe

### Second Embodiment

The current invention change in today's regular procedure is that before the above route the patient is injected with radiolabeled antibodies or radiolabeled Immunoscintigraphy pharmaceutical, which binds to inflammatory processes that characterize vulnerable plaque. Examples of such radiopharmaceuticals include Ga-67 Citrate, Tc-99m labeled white blood cells, F-18 DG (FDG), In-111 labeled white blood cells etc.

After the injection, the active tissue emits α, β⁻, β⁺, γ radiation with higher density compared with it surrounding, especially the non-active mass. The problem of poor resolution and lack of specificity is now reduced to locating the highest radiation source within the tissue by using radiation-timing detector inside the artery.

The timing detector is thread on a guiding wire to the end of the inspected artery.

The particle interaction inside the fiber scintillation produce two light groups that travels to the two ends of the scintillation fiber. Measuring the arrival time difference between the two light groups indicates which detector part has an active tissue. The present of the timing nuclear detector gives the ability to detect the vulnerable plaque artery parts and to treat them before they cases dangerous situations.

This method can also be applied to other illnesses, which absorb certain radiopharmaceuticals and use in vivo techniques.

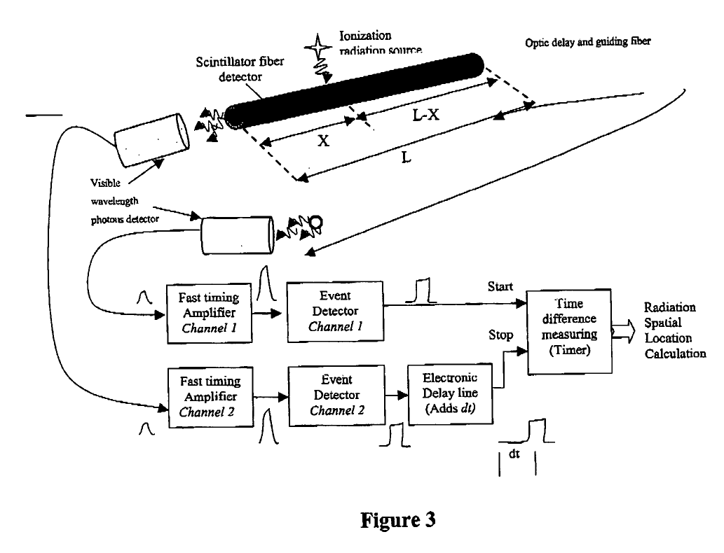

Referring further to the drawings, FIG. 3 schematically illustrates a general view of the detector inside the artery cross section and a timing coded detector block diagram, in accordance with the present invention.

The timing fiber detector composed of a polystyrene core doped with scintillating molecules and of fluoropolymer cladding. The fiber absorbs radiation all along its surface. A part of the radiation is transformed into photons with visible wavelength (red, blue, green etc) depending on the dopant. Because of the fiber structure the visible emitted photon (the light) are spited into two directions (the two fiber ends directions) and guided into the ends of the fiber. After reaching the scintillating fiber end, the light can possibly guided with regular optical fiber to a two sensitive photon detector or photo detector array such as:

Streak Camera made by Hamamatsu Japan models number such as: C6138 with time resolution of <300 fsec, C6860-M6861 with time resolution of <500 fsec, C5680 with time resolution of <2 psec, C5680-06 with time resolution of <5 psec, C2830 with time resolution of <10 psec, C6860-M6863 with time resolution of <50 psec.

Photo Multiplier Tube (PMT) made by Hamamatsu Japan (PMT) models number such as: R2496 with time resolution of <700 psec, R1894 with time resolution of <800 psec, R3991 with time resolution of <1 nsec.

Also possible to use Silicon Photo Diode (SPD) (with time resolution of <1 nsec), CCD, MCP etc.

Pulses from two sensitive photon detectors are connected in to two fast timing amplifiers. The amplifiers are fed into two event detectors (like Single Channel Analyzers (SCA), Snap Off etc . . . ). The event detectors provided logic pulses, lead into a measuring timer (like Time to Amplitude Converter (TAC), coincidence unit etc . . . ) that measure the arrival time deference between the light signal of the two fiber ends. Channel 1 and 2 event detectors detection level should be higher then the noise level in order to select true signals. By using the measured time difference the radiation source spatial location is calculated.

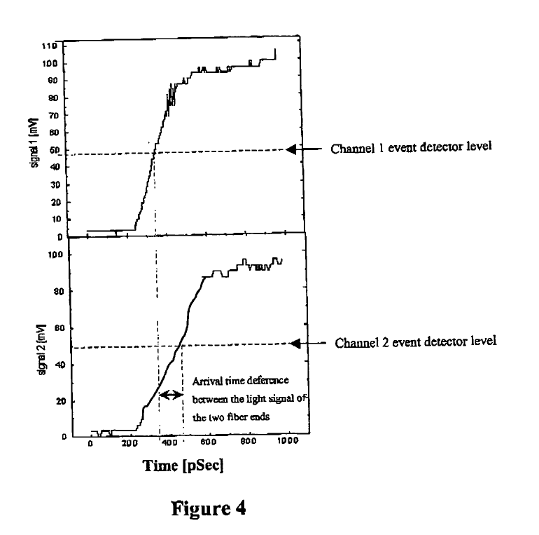

Referring further to the drawings, FIG. 4 schematically illustrates a light detector signal pair from the two fiber's ends, in accordance with the present invention.

Consider a radiation source that is impinging on the detector's side surface. This will allow light transfer to the two fiber ends through the entire fiber length L. If the radiation interaction point occurs at distance X from one of the fiber ends (Relative fiber's start) than one light group is traveling X distance while the other group is traveling L-X distance (To the relative fiber's end).

By using the speed of light C(C=˜3·10⁸ m/sec) the fiber traveling time of each fiber end can easily calculated by using Equation 1.

t_{end1}=(L−X)/C and t_{end2}=X/C Eq. 1

The arrival time difference dt between the two ends is calculated by using Equation 2.

dt=1/C·(L−2X) Eq. 2

By extracting the radiation interaction point from Equation 2 it can be calculated by using Equation 3.

X=(L−(dt·C))/2 Eq.3

For example if the measured time difference is dt=0 then by using Equation 3

X=L/2 measuring time difference (between the two light groups) of zero means that the to light groups traveled the same fiber length, this can only happened exactly in the middle of the fiber which mean L/2.

From the reason that is not possible to measure virtually negative arrival time difference between the two fiber ends an electronic or optic delay line should be added to make time offset such as if X=L/2 the measured arrival time difference should be dt=D.

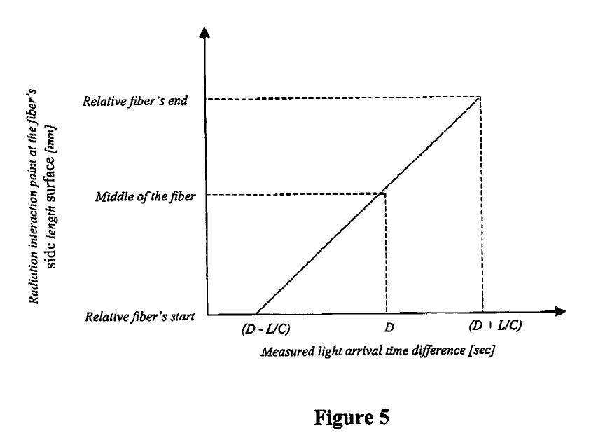

Referring further to the drawings, FIG. 5 schematically illustrates a radiation interaction point at the fiber's side length surface verses the measured light arrival time difference from the two fiber's ends, in accordance with the present invention.

Other way to measure the arrival time measurement can be done by using only one side of the scintillating detector. For this purpose the inner fiber's end (the inside examined body fiber's end) is covered with optical reflector (the reflector reflect all or nearly all of the light reaching to the fiber's end). If the radiation interaction point occurs at distance X from one of the fiber ends (Relative fiber's start) than one light group is traveling X distance while in this case the other group is traveling L-X distance (To the relative fiber's end) and because of the reflected fiber's end this light group is reflected and travels back to the fiber's relative start by traveling more L distance. All together the second group traveled 2L-X distance. By implying detection algorithm (clipping, SCA, Snap off etc . . . ) on the light output from the relative fiber's start (detected by a sensitive photon detector or photo detector array) the timing measurement can be achieved. Appling a wave shifter (shifts the wavelength to other wavelength) to the inner fiber's end assist signal difference detection and by this support the arrival time deference measurement.

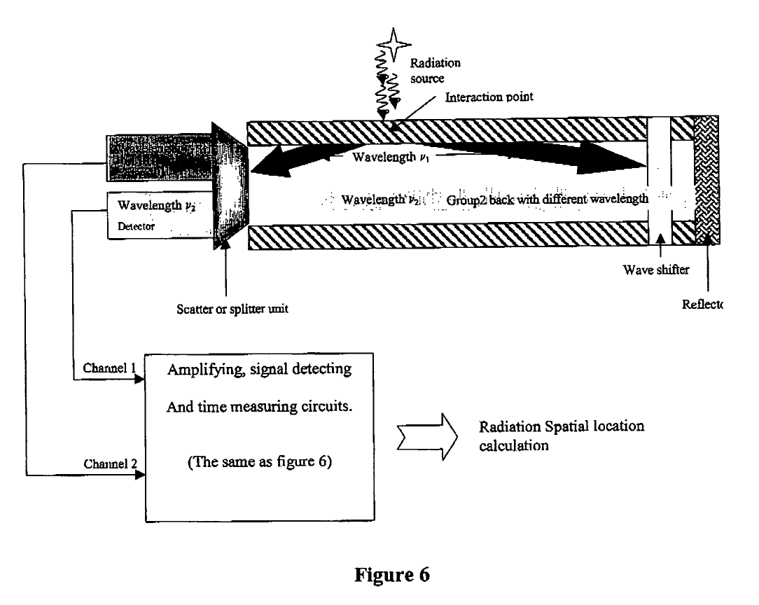

Referring further to the drawings, FIG. 6 schematically illustrates a fiber timing coded reflected detector's (cross-section) flow diagram, in accordance with the present invention.

Light arrival signal pair illustration measured simultaneously from the two fiber's ends is shown in FIG. 4 (one can see the measure arrival time difference between the two ends).

Improving the fiber position of interaction special resolution is made by performing deconvolution algorithm on the special information measured by a relative low timing resolution system. This can be done by incorporating a position sensor on the catheter (such as BIOSENSE WEBSTER LTD. magnetic position sensor) is pulled out slowly within the coronary lumen, the relative position of the sensor can be known and calculations can be made to reconstruct the radiation distribution based on algorithms described in Reference WO02-16965A2 For example, if the timing resolution is 100 Pico sec which gives 30 mm spatial resolution, then, by incorporating a position sensor and moving the catheter along the lumen, a super resolution algorithm such as in the above reference can be used to improve the spatial resolution by the super resolution factor, typically, 4 to 5 can be achieved. This can improve the spatial resolution from 30 mm to 5-6 mm. Since the intent is to use electrons as the radiation for detection, there is no need for collimation.

Another possibility is to use discrete radiation detectors or an array of color coded detectors as described below and improve on the spatial resolution in the same manner as described above.

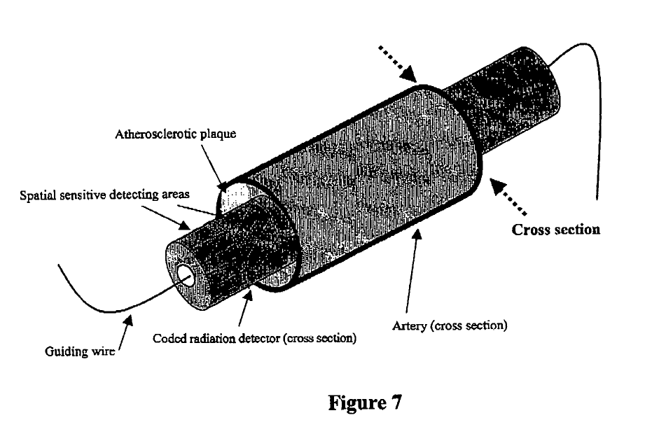

Referring further to the drawings, FIG. 7 schematically illustrates a general view of the detector inside the artery cross section and the coded detector inside the artery cross section, in accordance with the present invention.

Possible use of assembly is by using scintillating plastic optical fiber/s. These fiber/s composed of a polystyrene core doped with scintillating molecules and of fluoropolymer cladding. The fiber absorbs radiation all along its surface. A part of the radiation is transformed into photons with visible wavelength (red, blue, green etc) depending on the dopant. Then the visible photon guided at the end of the fiber and detected with sensitive photon detector such as Photo Multiplier Tube (PMT), Silicon Photo Diode (SPD), CCD, MCP etc.

Modifying and varying the dopant material types and their concentration along the fiber axis gives the ability to locate the radiation spatial source by performing wavelength spectrum analysis on the emitted fiber output light.

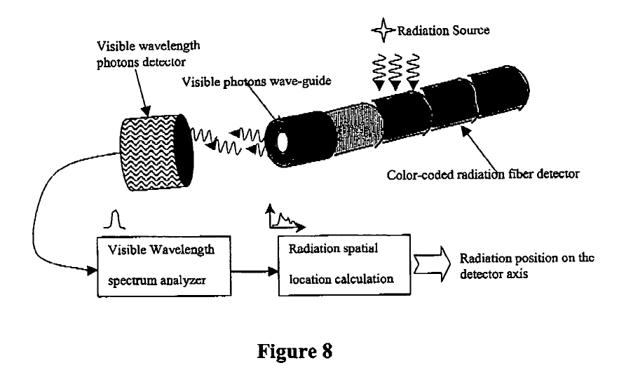

Referring further to the drawings, FIG. 8 schematically illustrates a signal processing flow block diagram, in accordance with the present invention.

Another way then using dopant material to manufacture the color-coded radiation fibers detectors is to use any kind of scintillator paint, scintillator powder, liquid scintillator etc. on a plastic, glass or any kind of optical fiber. The use scintillator paint, powder and liquid color is changed along the fiber axis for the detector localization coding.

Few color-coded radiation fibers detectors can be assembled as one detecting unit for 360° detecting with a guiding wire pass between them.

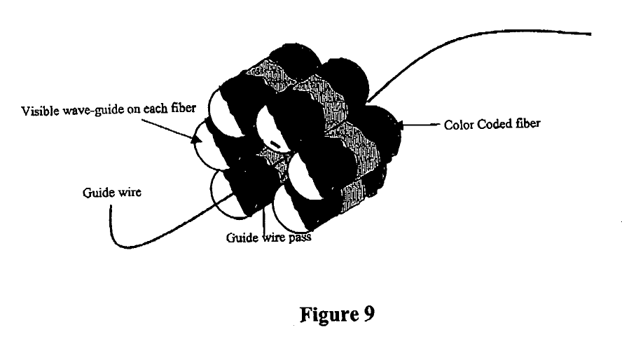

Referring further to the drawings, FIG. 9 schematically illustrates a multi fiber detector assembly cross section threaded on a guide wire, in accordance with the present invention.

The coded fiber itself can serve as a radiation detector and a guide wire. In this case, the coded fiber optic wire will be fitted with a flexible string at the distal end.

In this case, the guide wire will serve for all standard applications as well as give a real time intra lumen nuclear medicine image of the coronary arteries.

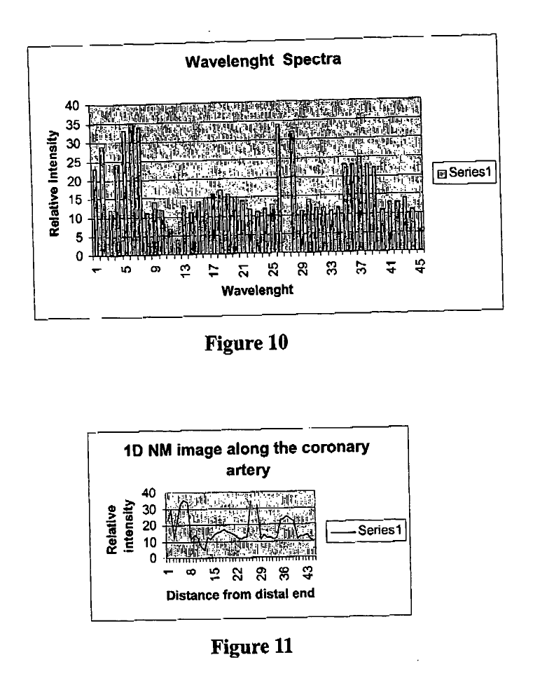

Referring further to the drawings, FIG. 10 is a wavelength spectra, in accordance with invention.

The ID image along the coronary lumen is calculated in the following way:

1. The color coded fiber optic is connected to a photo multiplier ( or photo diode etc) which converts the light signals into electrical signals.

2. The electrical signals are transferred into a spectrum analyzer whose output is ordered in color and the relative strength of the signal at the different photon wavelengths.

3. The output signal from the spectrum analyzer is decoded with an imaging algorithm such as an Anger algorithm or a deconvolution type algorithm. This algorithm calculates the relative intensity at different locations along the color coded optic fiber.

4. The result is a position vs. intensity ID graph indicating possible positions of Venerable plaque along the path of the color coded optic fiber.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications in printed or electronic form, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for imaging vulnerable plaque in coronary arteries, comprising:
placing an optical fiber, formed of a scintillation detector within an artery;
obtaining a light signal at a proximal end, with respect to an operator, said light signal being an indicator of a scintillation event;
obtaining a reflected light signal, reflecting from a distal end; and
obtaining a time differentiation between said two signals, so as to obtain the distance from the operator to the point of the scintillation event.
